(19) United States
(12) Patent Application Publication
(10) Pub. No.: US 2023/0414536 A1
(43) Pub. Date: Dec. 28, 2023

(54) CYCLOBENZAPRINE TREATMENT FOR POST-ACUTE SEQUELAE OF (SARS)-CoV-2 INFECTION (PASC)

(71) Applicant: Tonix Pharmaceuticals Holding Corp., Chatham, NJ (US)

(72) Inventor: Seth Lederman, South Dartmouth, MA (US)

(21) Appl. No.: 18/212,500

(22) Filed: Jun. 21, 2023

Related U.S. Application Data
(60) Provisional application No. 63/354,215, filed on Jun. 21, 2022.

(51) Int. Cl. A61K 31/137; A61P 29/00; A61P 31/14; A61K 47/26

(52) U.S. Cl. CPC A61K 31/137; A61P 29/00; A61P 31/14; A61K 47/26

(57) ABSTRACT

The present disclosure provides methods for treating Post-Acute Sequelae of Severe Acute Respiratory Syndrome (SARS)-CoV-2 infection (PASC) or one or more symptoms associated with said PASC, comprising administering to a subject in need or at risk thereof a pharmaceutical composition comprising a therapeutically effective amount of cyclobenzaprine or a pharmaceutically acceptable salt thereof and a pharmaceutically acceptable carrier.

### CROSS-REFERENCE TO RELATED APPLICATIONS

This application claims priority to and benefit from U.S. Provisional Application No. 63/354,215, filed Jun. 21, 2022, the contents of which are hereby incorporated by reference in its entirety.

### BACKGROUND

Cyclobenzaprine, or 3-(5H-dibenzola[a,d]cyclohepten-5-ylidene)-N,N-dimethyl-1 propanamine, was first approved by the U.S. Food and Drug Administration in 1977 for the treatment of acute muscle spasms of local origin (Katz and Dube. Clin Ther. 1988; 10(2):216-28). Subsequent studies have shown cyclobenzaprine to also be effective in the treatment of fibromyalgia syndrome, post-traumatic stress disorder (PTSD), generalized anxiety disorder (GAD) and depression.

Post-Acute Sequelae of (SARS)-CoV-2 Infection (PASC) (colloquially known as “long COVID” or “long haulers”) is a term used to describe a set of symptoms experienced by people with a history of probable or confirmed SARS-CoV-2 infection, usually 3 months from the onset of COVID-19 infection with symptoms that last for at least two months and cannot be explained by an alternative diagnosis. PASC symptoms span multiple organ systems, can occur within symptom clusters (i.e., neurologic, non-neurologic, and systemic) and may also fluctuate or relapse over time (Davis et al. EchnicalMedicine. 2021; 38:101019, Crook et al. BMJ. 2021; 374:n1648, Bierle et al. J Prim Care Community Health. 2021; 12:1-8, WHO 2021). The lack of a standardized definition of PASC makes it difficult to determine the exact epidemiology, incidence rates, and the impact of the condition on long-term disability. A conservative estimate based on data collected from numerous countries is that on average 30% of people who have had COVID-19 will experience PASC (Nalbandian et al. Nat Med. 2021; 27(4):601-15).

PASC is a multi-faceted condition affecting multiple body systems. Symptoms of PASC may be new onset (e.g., new onset of pain), following initial recovery from an acute or even mild COVID-19 episode, or persist from the initial illness. While the symptoms of PASC vary, pain, fatigue and sleep disturbances were found to be the main symptoms affecting quality of life and the ability to return to full time work (Alonso-Matielo et al. Front Physiol. 2021; 594 (7862):259-64, Davis et al. EchnicalMedicine. 2021; 38:101019, Sahin et al. Eur Neurol. 2021; 84:450-9). There is currently no FDA approved treatment for PASC. There is, therefore, an unmet need to reduce pain and other symptoms associated with PASC in this population. People suffering from PASC are generally treated with drugs targeting peripheral pain, including opioids, however, the central sensitization (or nociplastic) characteristic of multi-site pain in PASC suggests that there may be a lack of response to these treatments. Cyclobenzaprine HCl, is a non-opioid centrally acting analgesic that may provide a treatment for this unmet need of reducing pain and other symptoms in people suffering from PASC.

### SUMMARY

Some embodiments of this disclosure are:
1. A method for treating Post-Acute Sequelae of Severe Acute Respiratory Syndrome (SARS)-CoV-2 infection (PASC) or one or more symptoms associated with said PASC, comprising administering to a subject in need or at risk thereof a pharmaceutical composition comprising a therapeutically effective amount of cyclobenzaprine or a pharmaceutically acceptable salt thereof and a pharmaceutically acceptable carrier.
2. The method according to embodiment 1, wherein the pharmaceutically acceptable salt of cyclobenzaprine in the pharmaceutical composition is a cyclobenzaprine acid salt.
3. The method according to embodiment 2, wherein the cyclobenzaprineacid salt is cyclobenzaprine HCl.
4. The method according to any one of embodiments 1-3, wherein the cyclobenzaprine or pharmaceutically acceptable salt thereof is in the form of a eutectic.
5. The method according to embodiment 4, wherein the eutectic is a mannitol eutectic.
6. The method according to embodiment 5, wherein the mannitol eutectic is selected from the group consisting of a 75% 2% cyclobenzaprine HCl and 25% 2% β-mannitol eutectic, a 65%±2% cyclobenzaprine HCl and 35%±2% δ-mannitol eutectic, a mixture of a 75%±2% cyclobenzaprine HCl and 25%+2% β-mannitol and a 65%±2% cyclobenzaprine HCl and 35%±2% δ-mannitol eutectic, and a granule comprising an outer layer of a 65%+2% cyclobenzaprine HO and 35%+2% δ-mannitol eutectic and an inner layer of β-mannitol.
7. The method according to any one of embodiments 1-6, wherein the pharmaceutical composition comprising a pharmaceutically acceptable salt of cyclobenzaprine or the eutectic thereof further comprises a basifying agent.
8. The method according to embodiment 7, wherein the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIES buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.
9. The method according to embodiment 8, wherein the basifying agent is dipotassium hydrogen phosphate.
10. The method according to any one of embodiments 1-9, wherein the pharmaceutical composition comprises between 0.1 mg and 30 mg of cyclobenzaprine or a pharmaceutically acceptable salt thereof.
11. The method according to embodiment 10, wherein the pharmaceutical composition comprises between 1 mg and 20 mg of cyclobenzaprine or a pharmaceutically acceptable salt thereof
12. The method according to any one of embodiments 1-11, wherein the pharmaceutical composition comprises less than 10 mg of cyclobenzaprine or a pharmaceutically acceptable salt thereof
13. The method according to embodiment 12, wherein the pharmaceutical composition comprises less than 5 mg of cyclobenzaprine or a pharmaceutically acceptable salt thereof.
14. The method according to embodiment 12, wherein the pharmaceutical composition comprises about 5.6 mg of cyclobenzaprine HCl.
15. The method according to embodiment 12 or 13, wherein the pharmaceutical composition comprises about 2.8 mg of cyclobenzaprine HCl.
16. The method according to embodiment 12, wherein the pharmaceutical composition comprises between about 2.8 mg to about 5.6 mg of cyclobenzaprine HCl.
17. The method according to embodiment 14, wherein the pharmaceutical composition is administered simultaneously or sequentially in two dosage units, and wherein the combined amount of the cyclobenzaprine HCl in the two dosage units is about 5.6 mg.
18. The method according to embodiment 15, wherein the pharmaceutical composition is administered simultaneously in two dosage units, and wherein each dosage unit comprises about 2.8 mg of cyclobenzaprine HCl.
19, The method according to any one of embodiments 1-18, wherein the pharmaceutical composition is administered daily.
20. The method according to embodiment 19, wherein the pharmaceutical composition is administered once daily,
21. The method according to embodiment 19 or 20, wherein the pharmaceutical composition is administered at bedtime.
22. The method according to any one of embodiment 1-21, wherein the pharmaceutical composition is formulated for sublingual, buccal, intranasal, oral, intravenous, intramuscular, subcutaneous, inhalational, transdermal, rectal, vaginal, parenteral or palatal administration.
23. The method according to embodiment 22, wherein the pharmaceutical composition is formulated as a tablet, a thin film or a suppository.
24. The method according to embodiment 22, wherein the pharmaceutical composition is formulated for sublingual administration.
25. The method according to any one of embodiments 1-24, wherein the pharmaceutical composition is administered for at least 14 weeks.
26. The method according to any one of embodiments 1-25, wherein the subject has tested positive for SARS-CoV-2 infection at least three months prior to administration of the pharmaceutical composition.
27. The method according to embodiment 1, wherein the one or more symptoms associated with the PASC is neurologic, non-neurologic, systemic, or a combination thereof.
28. The method according to embodiment 1, wherein the one or more symptoms associated with the PASC is selected from the group consisting of fatigue, malaise, pain, muscle weakness, diaphoresis, chills, limb edema, dizziness, cognitive dysfunction, respiratory symptoms, cardiovascular abnormalities, alopecia, olfactory abnormalities, psychosocial symptoms, and abdominal symptoms.
29. The method according to embodiment 28, wherein the respiratory symptoms are independently selected from the group consisting of polypnea, chest pain, cough, sputum, sore throat, throat pain, abnormal breathing, and shortness of breath.
30. The method according to embodiment 28, wherein the cognitive dysfunction is characterized by brain fog.
31. The method according to embodiment 30, wherein the brain fog is one or more of a memory problem, a concentration problem, a lack of mental clarity, or an inability to focus.
32. The method according to embodiment 28, wherein the psychosocial symptoms are independently selected from the group consisting of sleep disturbance, depression, anxiety, feelings of inferiority, and worse quality of life.
33. The method according to embodiment 32, wherein the sleep disturbance is independently selected from the group consisting of insomnia, difficulty falling asleep, vivid or lucid dreams, and nonrestorative sleep.
34. The method according to embodiment 28, wherein the malaise is post-exertional malaise.
35. The method according to embodiment 28, wherein the pain is independently selected from the group consisting of multi-site pain, diffuse myalgia, arthralgia, musculoskeletal pain, headaches, facial pain, chest pain, abdominal pain, back pain, joint pain, body ache, lumbago with sciatica, low back pain, and pain in one or more of limb, hand, foot fingers, or toes.
36. The method according to embodiment 35, wherein the one or more symptoms associated with the PASC is multi-site pain.
37. The method according to embodiment 28 or 36, wherein the one or more symptoms associated with the PASC are multi-site pain and fatigue.
38. The method according to embodiment 33 or 36, wherein the one or more symptoms associated with the PA SC are multi-site pain and insomnia.
39. The method according to any one of embodiments 28, 33 and 36, wherein the one or more symptoms associated with the PASC are multi-site pain, fatigue, and insomnia.
40. The method according to any one of embodiments 35-39, wherein multi-site pain affects at least 4 regions of the body.
41. The method according to embodiment 40, wherein the multi-site pain regions are assessed using a Michigan Body Map.
42. The method according to embodiment 41, wherein the multi-site pain region is selected from one or more of the regions of a Michigan Body Map including left arm, right arm, left leg, right leg, front of trunk, back of trunk, or head.
43. The method according to any one of embodiments 27-42, wherein the one or more symptoms associated with the PASC is new onset, follows initial recovery from an acute (SARS)-CoV-2 infection, persists post-(SARS)-CoV-2 infection, or persists post-discharge from in-patient care in a hospital, clinic or other medical facility following admission for (SARS)-CoV-2 infection.
44. The method according to embodiment 43, wherein the one or more symptoms associated with the PASC fluctuates or relapses over time.
45. The method according to embodiment 43 or 44, wherein the one or more symptoms associated with the PASC persists post-(SARS)-CoV-2 infection.
46. The method according to embodiment 45, wherein the one or more symptoms associated with the PASC persists for at least 2 months post-(SARS)-CoV-2 infection.
47. The method according to embodiment 46, wherein the one or more symptoms associated with the PASC persists for about 8-12 weeks post-(SARS)-CoV-2 infection.
48. The method according to embodiment 46, wherein the one or more symptoms associated with the PASC persists for about 3-18 months post-(SARS)-CoV-2 infection.
49. The method according to embodiment 46, wherein the one or more symptoms associated with the PASC persists for about 90 days post-(SARS)-CoV-2 infection.
50. The method according to embodiment 46 or 48, wherein the one or more symptoms associated with the PASC persists up to about 18 months post-(SARS)-CoV-2
51. The method according to embodiment 46 or 48, wherein the one or more symptoms associated with the PASC persists for about 6 months post-(SARS)-CoV-2 infection.
52. The method according to embodiment 43 or 44, wherein the one or more symptoms associated with the PASC persists post-discharge from in-patient care in hospital, clinic or other medical facility following admission for (SARS)-CoV-2 infection.
53. The method according to embodiment 52, wherein the one or more symptoms associated with the PASC persists about 60 days post-discharge from in-patient care in a hospital, clinic or other medical facility following admission for (SARS)-CoV-2 infection.
54. The method according to any one of embodiments 1 to 53, wherein the one or more symptoms associated with the PASC is assessed by a Numerical Rating Scale (NRS), a Patient Global Impression of Change (PGI-C), a PROMIS scale, a Sheehan Disability Scale (SUS), a Post-COVID-19 Functional Status (PCFS) scale, an Insomnia Severity Index (ISI), an Epworth Sleepiness Scale (ESS), or a combination thereof.
55. The method according to embodiment 54, wherein the PROWS scale is selected from the group consisting of a PROMIS-Sleep disturbance scale, a PROMIS-Fatigue scale, and a PROMIS-Cognitive function scale.
56. The method according to any one of embodiments 1 to 55, Therein the subject is human.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows the Michigan Body Map (MBM) used to assess widespread pain.

FIG. 2 shows the diagnostic codes used to select subjects with multi-site pain. Any COVID-19-related diagnosis code (ICD-10-CM) or positive PCR test (LONIC®) occurring on or after Jan. 20, 2020. Excluding those with diagnosis of other specified viral infection (code 879.89) on or after Jan. 20, 2020.¹ Multi-site pain include: myalgia, myositis, fibromyalgia, pain or >2 other unique pain diagnosis codes in the time period of interest. Pain in joint, limb, hand, foot, fingers, toes, throat, and chest were evaluated using the specific child within the parent code.² [COVID=coronavirus disease; LONIC=Logical Observational Identifiers Names and Codes]

FIGS. 3A and 3B are tables showing the demographic of subjects with COVID-19 and PASC (FIG. 3A) and PASC and multi-site pain (FIG. 3B).

FIG. 4 is a table showing the prevalence of pain-related diagnoses for multi-site pain in subjects with PASC.

FIG. 5 is a graph showing the prevalence of breathing abnormalities, abdominal symptoms, anxiety/depression and cognitive symptoms (or “brain fog”) in subjects with PASC at days 91-180.

FIG. 6 is a graph showing the prevalence of inflammatory markers (e.g., erythrocyte sedimentation rate and C-Reactive protein) and tissue damage markers (e.g., alkaline phosphatase and creatine kinase) in subjects with PASC.

FIG. 7 is a graph showing the prevalence of PASC subjects that use analgesics, NSAIDS, anti-inflammatories, and sedatives/hypnotics. [NSAID=Non-steroidal anti-inflammatory drug]

FIG. 8 is a graph showing the prevalence of PASC subjects that use benzodiazepine derivative anti-anxiolytics, opioids and non-opioids.

### DETAILED DESCRIPTION

The present disclosure provides in some embodiments, methods and pharmaceutical compositions for treating Post-Acute Sequelae of (SARS)-CoV-2 Infection (PASC) or one or more symptoms associated therewith in a subject in need or at risk thereof, wherein the pharmaceutical compositions comprise a therapeutically effective amount of cyclobenzaprine or a pharmaceutically acceptable salt thereof and a pharmaceutically acceptable carrier. In other embodiments, the present disclosure provides the use of cyclobenzaprine or a pharmaceutically acceptable salt thereof in the preparation of a medicament for treating PASC or one or symptoms associated therewith.

### General

The term “herein” means the entire application.

Unless otherwise defined herein, scientific and technical terms used in this application shall have the meanings that are commonly understood by those of ordinary skill in the art. In case of conflict, the present specification, including definitions, will control.

It should be understood that any of the embodiments described herein, including those described under different aspects of the disclosure and different parts of the specification (including embodiments described only in the Examples) can be combined with one or more other embodiments of this disclosure, unless explicitly disclaimed or improper, and are so disclosed as embodiments to the disclosure. Combination of embodiments are not limited to those specific combinations described in the multiple dependent embodiments of this disclosure.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, the term “about” refers to a value or parameter that includes (and describes) embodiments that are directed to that value or parameter per se. For example, description referring to “about X” includes description of “X”. As used herein, the term “about” permits a variation of ±10% within the range of the significant digit. Numeric ranges are inclusive of the numbers defining the range.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members.

Exemplary methods and materials are described herein, although methods and materials similar or equivalent to those described herein can also be used in the practice or testing of the various aspects and embodiments. The materials, methods, and examples are illustrative only and not intended to be limiting.

### Definitions

In order that the disclosure may be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “treat” and its cognates refer to a full or partial amelioration or modulation of Post-Acute Sequelae of Severe Acute Respiratory Syndrome (SARS)-CoV-2 infection (PASC) or at least one discernible symptom associated therewith with cyclobenzaprine, a pharmaceutically acceptable salt of cyclobenzaprine, or a composition comprising cyclobenzaprine or the pharmaceutically acceptable salt of cyclobenzaprine and a pharmaceutically acceptable carrier. In some embodiments, “treat” refers to a reduction of pain. In some embodiments, “treat” refers to reduction of sleep disturbance. In some embodiments, “treat” refers to an improvement in sleep quality. In some embodiments, “treat” refers to a reduction of fatigue. In some embodiments, “treat” refers to improved concentration. In some embodiments, “treat” refers to “improved,” “much improved,” or “very much improved” in the context of these and other symptoms associated with PASC.

In some embodiments, the cyclobenzaprine is in the form of the free base or a pharmaceutically acceptable salt of the free base. In some embodiments, the cyclobenzaprine is the free base. In some embodiments, the cyclobenzaprine is a pharmaceutically acceptable salt. In some embodiments, the cyclobenzaprine is an acid salt. In some embodiments, the cyclobenzaprine acid salt is cyclobenzaprine hydrochloride (cyclobenzaprine HCl) (See e.g., WO2013/188847, incorporated herein by reference).

In some embodiments of this disclosure, the cyclobenzaprine or its acid salt is present in a eutectic. In some embodiments, the eutectic includes mannitol. In other embodiments, the mannitol is beta-mannitol or delta-mannitol. In some embodiments the cyclobenzaprine HCl is in a form of a eutectic selected from the group consisting of a 75%±2% cyclobenzaprine HCl and 25%±2% β-mannitol eutectic, a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%±2% δ-mannitol eutectic and an inner layer of β-mannitol. See, e.g., WO2014/145156 and WO2016/044796, both incorporated herein by reference. It should be understood that the “cyclobenzaprine HCl” eutectic of this disclosure refers to any of these eutectics or granules.

As used herein, the term a “eutectic” or “in the form of a eutectic” refers to a mixture of chemical compounds or elements that has a single chemical composition that melts at a lower temperature than any other composition made up of the same ingredients. A composition comprising a eutectic is known as a eutectic composition and its melting temperature is known as the eutectic temperature. Eutectic compositions often have a higher stability and/or dissolution rates than their non-eutectic counterparts. Because eutectics enhance dissolution, they can be employed to increase permeability in solid dispersions and dispersion systems.

In some embodiments, the pharmaceutical composition comprises a pharmaceutically acceptable salt of cyclobenzaprine or eutectic of a pharmaceutically acceptable salt of cyclobenzaprine and a basifying agent. In some embodiments, the basifying agent exerts its effects during the time the formulation is being dispersed in the mucous material, including buccal and sublingual tissue, while parts of the formulation are dissolving in the mucous material and for a period of time after the tablet is dissolved in the mucous material.

As used herein, “basifying agent” is selected from a group consisting of potassium dihydrogen phosphate (monopotassium phosphate, monobasic potassium phosphate, KH₂PO₄), dipotassium hydrogen phosphate (dipotassium phosphate, dibasic potassium phosphate, K₂HPO₄), tripotassium phosphate (K₃PO₄), sodium dihydrogen phosphate (monosodium phosphate, monobasic sodium phosphate, NaH₂PO₄), disodium hydrogen phosphate (disodium phosphate, dibasic sodium phosphate, Na₂HPO₄), trisodium phosphate (Na₃PO₄), bicarbonate or carbonate salts, dipotassium citrate, tripotassium citrate, disodium citrate, trisodium citrate, borate, hydroxide, silicate, nitrate, dissolved ammonia, the conjugate bases of some organic acids (including bicarbonate), and sulfide. A basifying agent with particular effects on cyclobenzaprine HCl is dipotassium hydrogen phosphate (K₂HPO₄). Another basifying agent with particular effects on cyclobenzaprine HCl is potassium dihydrogen phosphate (KH₂PO₄). Another basifying agent with particular effects on cyclobenzaprine HCl is disodium hydrogen phosphate (Na₂HPO₄). Another basifying agent with particular effects on cyclobenzaprine HCl is tripotassium citrate. Another basifying agent with particular effects on cyclobenzaprine HCl is trisodium citrate.

As used herein, “TNX-102 SL” refers to a low dose, sublingual formulation of a cyclobenzaprine HCl-mannitol eutectic and a basifying agent, as described in PCT Application No. WO2013/188847, which is incorporated herein by reference. TNX-102 SL allows transmucosal absorption of the cyclobenzaprine free base into the blood, and without wishing to be bound by theory, uniquely reduces production of a long half-life active metabolite of cyclobenzaprine, norcyclobenzaprine, due to its bypass of first-pass hepatic metabolism. This allows much improved long-term efficacy.

As used herein, “subject” and “patient” are used interchangeably herein and refer to mammals including, but not limited to, human and non-human animals. These terms include mammals, such as humans, and primates (e.g., monkey, gorilla, ape, and chimpanzee). In some embodiments, the subject is a human. Accordingly, the term “subject” or “patient” as used herein means any mammalian patient or subject to which the compositions of the disclosure may be administered. In some embodiments, the subject is in need of treatment of PASC or one or more symptoms associated with PASC.

As used herein, “multi-site pain” or “multi-site pain associated with PASC” refers to persisting pain in 4 or more regions on the Michigan Body Map. These regions on the Michigan Body map include one or more of left arm, right arm, left leg, right leg, front of trunk, back of trunk, or head.

### Post-Acute Sequelae of Severe Acute Respiratory Syndrome (Sars)-CoV-2 Infection (PASC)

Post-Acute Sequelae of (Sars)-CoV-2 Infection (PASC) (colloquially known as “Long COVID” or “long haulers”) occurs in individuals with a history of probable or confirmed SARS-CoV-2 infection, usually 3 months from the onset of SARS-CoV-2 infection, with symptoms that last for at least 2 months, and often more, and cannot be explained by an alternative diagnosis. PASC can broadly be described as the presence of one or more symptoms (continuous or relapsing/remitting; new or same symptoms of acute COVID-19) in individuals who have been infected with SARS-CoV-2 even after the clinical period of clinical recovery from acute disease. One or more symptoms associated with PASC include fatigue, muscle weakness, diaphoresis, myalgia, arthralgia, chills, limb edema, dizziness, post-exertional malaise, cognitive dysfunction, respiratory symptoms (polypnea, chest pain, cough, sputum, sore throat), cardiovascular abnormalities, alopecia, olfactory abnormalities, neurocognitive difficulties including memory and concentration problems, psychological symptoms such as sleep difficulties, depression, anxiety, feelings of inferiority, and generally a worse quality of life.

Pain, fatigue and sleep disturbances were found to be the main symptoms associated with PASC affecting quality of life. Fatigue occurred in roughly 40% to 80% of PASC patients at an average of 4 to 8 weeks post infection (Crook et al. BMJ 2021; 374:n1648, Davis et al. EChnicalMedicine. 2021; 38:101019, Lambert et al. medRxiv. 2021, Lopez-Leon et al. medRxiv. 2021, Bierle et al. J Prim Care Community Health. 2021; 12:1-8). Approximately 20% to 64% of patients also report persistent pain up to 8 to 12 weeks beyond the resolution of acute viral infection (Moreno-Perez et al. J Infect. 2021; 82(3):378-83, Lambert et al. medRxiv. 2021). The types of pain reported in PASC include diffuse myalgias, arthralgias, musculoskeletal pain, headaches chest pain, abdominal pain and generalized “body ache.” In many case, multiple sites of pain were reported. A variety of sleep disturbances have also been reported in PASC, with nearly 80% of PASC patients experiencing insomnia, difficulty falling asleep, vivid/lucid dreams or nonrestorative sleep (Davis et al. EChnicalMedicine. 2021; 38:101019).

### Method of Treating

In one aspect, the present disclosure relates to a method for treating Post-Acute Sequelae of Severe Acute Respiratory Syndrome (SARS)-CoV-2 infection (PASC) or one or more symptoms associated with said PASC comprising administering to a subject in need or at risk thereof a pharmaceutical composition comprising a therapeutically effective amount of cyclobenzaprine or a pharmaceutically acceptable salt of cyclobenzaprine and a pharmaceutically acceptable carrier.

In some embodiments, the pharmaceutically acceptable salt of cyclobenzaprine in the pharmaceutical composition used in the methods of this disclosure is a cyclobenzaprine acid salt. In some embodiments, the cyclobenzaprine acid salt used in the methods of this disclosure is cyclobenzaprine HCl.

In some embodiments, the cyclobenzaprine or pharmaceutically acceptable salt thereof used in the methods of this disclosure is in the form of a eutectic. In some embodiments, the pharmaceutically acceptable salt of cyclobenzaprine used in this disclosure is in the form of a eutectic. In some embodiments, the eutectic used in the methods of this disclosure is a mannitol eutectic. In some embodiments, the mannitol eutectic used in the methods of this disclosure is selected from the group consisting of a 75%±2% cyclobenzaprine HCl and 25%±2% β-mannitol eutectic, a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%±2% δ-mannitol eutectic and an inner layer of β-mannitol. In some embodiments, the mannitol eutectic used in the methods of this disclosure is a 75%±2% cyclobenzaprine HCl and 25%±2% β-mannitol eutectic. In some embodiments, the mannitol eutectic used in the methods of this disclosure is a 65%±2% cyclobenzaprine HCl and 35%±2% δ-mannitol eutectic. In some embodiments, the mannitol eutectic used in the methods of this disclosure is a mixture of a 75%±2% cyclobenzaprine HCl and 25%±2% β-mannitol and a 65%±2% cyclobenzaprine HCl and 35%±2% δ-mannitol eutectic. In some embodiments, the mannitol eutectic used in the methods of this disclosure is a granule comprising an outer layer of a 65%±2% cyclobenzaprine HCl and 35%±2% δ-mannitol eutectic and an inner layer of β-mannitol.

In another aspect, the present disclosure relates to a method for treating PASC or one or more symptoms associated with said PASC comprising administering a pharmaceutical composition comprising a pharmaceutically acceptable acid salt of cyclobenzaprine and a basifying agent. In some embodiments, the pharmaceutical composition comprising a pharmaceutically acceptable salt of cyclobenzaprine used in the methods of this disclosure is in the form of a eutectic and is administered with a basifying agent. In some embodiments, the basifying agent used in the methods of this disclosure is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate. In some embodiments, the basifying agent used in the methods of this disclosure is potassium dihydrogen phosphate. In some embodiments, the basifying agent used in the methods of this disclosure is dipotassium hydrogen phosphate. In some embodiments, the basifying agent used in the methods of this disclosure is tripotassium phosphate. In some embodiments, the basifying agent used in the methods of this disclosure is sodium carbonate. In some embodiments, the basifying agent used in the methods of this disclosure is sodium bicarbonate. In some embodiments, the basifying agent used in the methods of this disclosure is calcium carbonate. In some embodiments, the basifying agent used in the methods of this disclosure is calcium bicarbonate. In some embodiments, the basifying agent used in the methods of this disclosure is TRIS buffer. In some embodiments, the basifying agent used in the methods of this disclosure is sodium dihydrogen phosphate. In some embodiments, the basifying agent used in the methods of this disclosure is disodium hydrogen phosphate. In some embodiments, the basifying agent used in the methods of this disclosure is trisodium phosphate. In some embodiments, the basifying agent used in the methods of this disclosure is potassium carbonate. In some embodiments, the basifying agent used in the methods of this disclosure is potassium bicarbonate. In some embodiments, the basifying agent used in the methods of this disclosure is potassium acetate. In some embodiments, the basifying agent used in the methods of this disclosure is sodium acetate. In some embodiments, the basifying agent used in the methods of this disclosure is dipotassium citrate. In some embodiments, the basifying agent used in the methods of this disclosure is tripotassium citrate. In some embodiments, the basifying agent used in the methods of this disclosure is disodium citrate. In some embodiments, the basifying agent used in the methods of this disclosure is trisodium citrate.

In some embodiments, the pharmaceutical composition used in the methods of this disclosure comprises between 0.1 mg and 30 mg of cyclobenzaprine or a pharmaceutically acceptable salt thereof. In some embodiments, the pharmaceutical composition used in the methods of this disclosure comprises between 1 mg and 20 mg of cyclobenzaprine or a pharmaceutically acceptable salt thereof. In some embodiments, the pharmaceutical composition used in the methods of this disclosure comprises less than 10 mg of cyclobenzaprine or a pharmaceutically acceptable salt thereof. In some embodiments, the pharmaceutical composition used in the methods of this disclosure comprises less than 5 mg of cyclobenzaprine or a pharmaceutically acceptable salt thereof. In some embodiments, the pharmaceutical composition used in the methods of this disclosure comprises about 5.6 mg of cyclobenzaprine HCl. In some embodiments, the pharmaceutical composition used in the methods of this disclosure comprises 5.6 mg of cyclobenzaprine HCl. In some embodiments, the pharmaceutical composition used in the methods of this disclosure comprises about 2.8 mg of cyclobenzaprine HCl. In some embodiments, the pharmaceutical composition used in the methods of this disclosure comprises 2.8 mg of cyclobenzaprine HCl. In some embodiments, the pharmaceutical composition used in the methods of this disclosure comprises between about 2.8 mg and about 5.6 mg of cyclobenzaprine HCl. In some embodiments, the pharmaceutical composition used in the methods of this disclosure comprises between 2.8 mg and 5.6 mg of cyclobenzaprine HCl.

In some embodiments, the pharmaceutical composition used in the methods of this disclosure comprises a therapeutically effective amount of cyclobenzaprine or a pharmaceutically acceptable salt of cyclobenzaprine and a pharmaceutically acceptable carrier, wherein the pharmaceutical composition is administered simultaneously or sequentially in two dosage units, wherein the combined amount of the cyclobenzaprine HCl in the two dosage units is about 5.6 mg. In some embodiments, the pharmaceutical composition used in the methods of this disclosure comprises a therapeutically effective amount of cyclobenzaprine or a pharmaceutically acceptable salt of cyclobenzaprine and a pharmaceutically acceptable carrier, wherein the pharmaceutical composition is administered simultaneously or sequentially in two dosage units, wherein the combined amount of the cyclobenzaprine HCl in the two dosage units is 5.6 mg. In some embodiments, the pharmaceutical composition used in the methods of this disclosure is administered simultaneously in two dosage units, wherein each dosage unit comprises about 2.8 mg of cyclobenzaprine HCl. In some embodiments, the pharmaceutical composition used in the methods of this disclosure is administered simultaneously in two dosage units, wherein each dosage unit comprises 2.8 mg of cyclobenzaprine HCl. In some embodiments, the pharmaceutical composition used in the methods of this disclosure is administered in a single dosage unit comprising about 2.8 mg of cyclobenzaprine HCl. In some embodiments, the pharmaceutical composition used in the methods of this disclosure is administered in a single dosage unit comprising 2.8 mg of cyclobenzaprine HCl. In some embodiments, the pharmaceutical composition used in the methods of this disclosure is administered daily. In some embodiments, the pharmaceutical composition used in the methods of this disclosure is administered once daily. In some embodiments, the pharmaceutical composition used in the methods of this disclosure is administered at bedtime. In some embodiments, the pharmaceutical composition used in the methods of this disclosure is administered for at least 14 weeks.

In some embodiments, the pharmaceutical composition or eutectic thereof used in the methods of this disclosure is formulated for sublingual, buccal, intranasal, oral, intravenous, intramuscular, subcutaneous, inhalational, transdermal, rectal, vaginal, parenteral or palatal administration. In some embodiments, the pharmaceutical composition used in the methods of this disclosure is formulated for sublingual administration. In some embodiments, the pharmaceutical composition used in the methods of this disclosure is formulated for buccal administration. In some embodiments, the pharmaceutical composition used in the methods of this disclosure is formulated for intranasal administration. In some embodiments, the pharmaceutical composition used in the methods of this disclosure is formulated for oral administration. In some embodiments, the pharmaceutical composition is used in the methods of this disclosure formulated for intravenous administration. In some embodiments, the pharmaceutical composition used in the methods of this disclosure is formulated for intramuscular administration. In some embodiments, the pharmaceutical composition used in the methods of this disclosure is formulated for subcutaneous administration. In some embodiments, the pharmaceutical composition used in the methods of this disclosure is formulated for inhalational administration. In some embodiments, the pharmaceutical composition used in the methods of this disclosure is formulated for transdermal administration. In some embodiments, the pharmaceutical composition used in the methods of this disclosure is formulated for rectal administration. In some embodiments, the pharmaceutical composition used in the methods of this disclosure is formulated for vaginal administration. In some embodiments, the pharmaceutical composition used in the methods of this disclosure is formulated for parenteral administration. In some embodiments, the pharmaceutical composition used in the methods of this disclosure is formulated for palatal administration.

In some embodiments, the pharmaceutical composition used in the methods of this disclosure is formulated as a tablet, a thin film or a suppository. In some embodiments, the pharmaceutical composition used in the methods of this disclosure is formulated as a tablet. In some embodiments, the pharmaceutical composition used in the methods of this disclosure is formulated as a thin film. In some embodiments, the pharmaceutical composition used in the methods of this disclosure is formulated as a suppository. In some embodiments, the pharmaceutical composition used in the methods of this disclosure is formulated as a tablet for sublingual administration. In some embodiments, the pharmaceutical composition used in the methods of this disclosure is formulated as a thin film for sublingual administration.

In some embodiments, the subject has tested positive for SARS-CoV-2 infection at least three months prior to administration of the pharmaceutical composition used in the methods of this disclosure.

In some embodiments, the one or more symptoms associated with the PASC is neurologic, non-neurologic, systemic, or a combination thereof. In some embodiments, the one or more symptoms associated with the PASC is neurologic. In some embodiments, the one or more symptoms associated with the PASC is non-neurologic. In some embodiments, the one or more symptoms associated with the PASC is systemic. In some embodiments, the one or more symptoms associated with the PASC is selected from the group consisting of fatigue, malaise, pain, muscle weakness, diaphoresis, chills, limb edema, dizziness, cognitive dysfunction, respiratory symptoms, cardiovascular abnormalities, alopecia, olfactory abnormalities, psychosocial symptoms, and abdominal symptoms. In some embodiments, the respiratory symptoms are independently selected from the group consisting of polypnea, chest pain, cough, sputum, sore throat, throat pain, abnormal breathing, and shortness of breath. In some embodiments, the cognitive dysfunction is characterized by brain fog. In some embodiments, the brain fog is one or more of a memory problem, a concentration problem, a lack of mental clarity or an inability to focus. In some embodiments, the psychosocial symptoms are independently selected from the group consisting of sleep disturbance, depression, anxiety, feelings of inferiority, and worse quality of life. In some embodiments, the sleep disturbance is independently selected from the group consisting of insomnia, difficulty falling asleep, vivid or lucid dreams, and nonrestorative sleep. In some embodiments, the malaise is post-exertional malaise. In some embodiments, the pain is independently selected from the group consisting of multi-site pain, diffuse myalgia, arthralgia, musculoskeletal pain, headaches, facial pain, chest pain, abdominal pain, back pain, joint pain, body ache, lumbago with sciatica, low back pain, and pain in limb, hand, foot fingers, and toes.

In some embodiments, the one or more symptoms associated with the PASC is selected from the group consisting of multi-site pain, fatigue, and insomnia. In some embodiments, the one or more symptoms associated with the PASC is multi-site pain. In some embodiments, the one or more symptoms associated with the PASC is multi-site pain but not insomnia or fatigue. In some embodiments, the one or more symptoms associated with the PASC are multi-site pain and fatigue. In some embodiments, the one or more symptoms associated with the PASC are multi-site pain and fatigue but not insomnia. In some embodiments, the one or more symptoms associated with the PASC are multi-site pain and insomnia. In some embodiments, the one or more symptoms associated with the PASC are multi-site pain and insomnia but not fatigue. In some embodiments, the one or more symptoms associated with the PASC is multi-site pain, fatigue, and insomnia. In some embodiments, the multi-site pain affects at least 4 regions of the body. In some embodiments, the multi-site pain regions are assessed using a Michigan Body Map. In some embodiments, the multi-site pain region is selected from one or more of the regions of a Michigan Body Map including left arm, right arm, left leg, right leg, front of trunk, back of trunk, or head. In some embodiments, the multi-site pain region is left arm. In some embodiments, the multi-site pain region is right arm. In some embodiments, the multi-site pain region is left leg. In some embodiments, the multi-site pain region is right leg. In some embodiments, the multi-site pain region is front of trunk. In some embodiments, the multi-site pain region is back of trunk. In some embodiments, the multi-site pain region is head.

In some embodiments, the one or more symptoms associated with the PASC is new onset, follows initial recovery from an acute (SARS)-CoV-2 infection, persists post-(SARS)-CoV-2 infection, or persists post-discharge from in-patient care in a hospital, clinic or other medical facility following admission for (SARS)-CoV-2 infection. In some embodiments, the one or more symptoms associated with the PASC is new onset. In some embodiments, the one or more symptoms associated with the PASC follows initial recovery from an acute (SARS)-CoV-2 infection. In some embodiments, the one or more symptoms associated with the PASC persists post-(SARS)-CoV-2 infection. In some embodiments, the one or more symptoms associated with the PASC persists for at least 2 months post-(SARS)-CoV-2 infection. In some embodiments, the one or more symptoms associated with the PASC persists for about 8-12 weeks post-(SARS)-CoV-2 infection. In some embodiments, the one or more symptoms associated with the PASC persists for about 3-18 months post-(SARS)-CoV-2 infection. In some embodiments, the one or more symptoms associated with the PASC persists for about 90 days post-(SARS)-CoV-2 infection. In some embodiments, the one or more symptoms associated with the PASC persists up to about 18 months post-(SARS)-CoV-2 infection. In some embodiments, the one or more symptoms associated with the PASC persists for about 6 months post-(SARS)-CoV-2 infection. In some embodiments, the one or more symptoms associated with the PASC persists post-discharge from in-patient care in a hospital, clinic or other medical facility following admission for (SARS)-CoV-2 infection. In some embodiments, the one or more symptoms associated with the PASC persists about 60 days post-discharge from in-patient care in a hospital, clinic or other medical facility following admission for (SARS)-CoV-2 infection. In some embodiments, the one or more symptoms associated with the PASC fluctuates or relapses over time.

In some embodiments, the one or more symptoms associated with the PASC is assessed by a Numerical Rating Scale (NRS), a Patient Global Impression of Change (PGI-C), a PROMIS scale, a Sheehan Disability Scale (SDS), a Post-COVID-19 Functional Status (PCFS) scale, an Insomnia Severity Index (ISI), an Epworth Sleepiness Scale (ESS), or a combination thereof. In some embodiments, the one or more symptoms associated with the PASC is assessed by a Numerical Rating Scale (NRS). In some embodiments, the one or more symptoms associated with the PASC is assessed by a Patient Global Impression of Change (PGI-C). In some embodiments, the one or more symptoms associated with the PASC is assessed by a PROMIS scale. In some embodiments, the one or more symptoms associated with the PASC is assessed by a Sheehan Disability Scale (SDS). In some embodiments, the one or more symptoms associated with the PASC is assessed by a post-COVID-19 Functional Status (PCFS) scale. In some embodiments, the one or more symptoms associated with the PASC is assessed by an Insomnia Severity Index (ISI). In some embodiments, the one or more symptoms associated with the PASC is assessed by an Epworth Sleepiness Scale (ESS). In some embodiments, the PROMIS scale is selected from the group consisting of a PROMIS-Sleep disturbance scale, a PROMIS-Fatigue scale, and a PROMIS-Cognitive function scale. In some embodiments, the PROMIS scale is a PROMIS-Sleep disturbance scale. In some embodiments, the PROMIS scale is a PROMIS-Fatigue scale. In some embodiments, the PROMIS scale is a PROMIS-Cognitive function scale.

### Michigan Body Map (MBM)

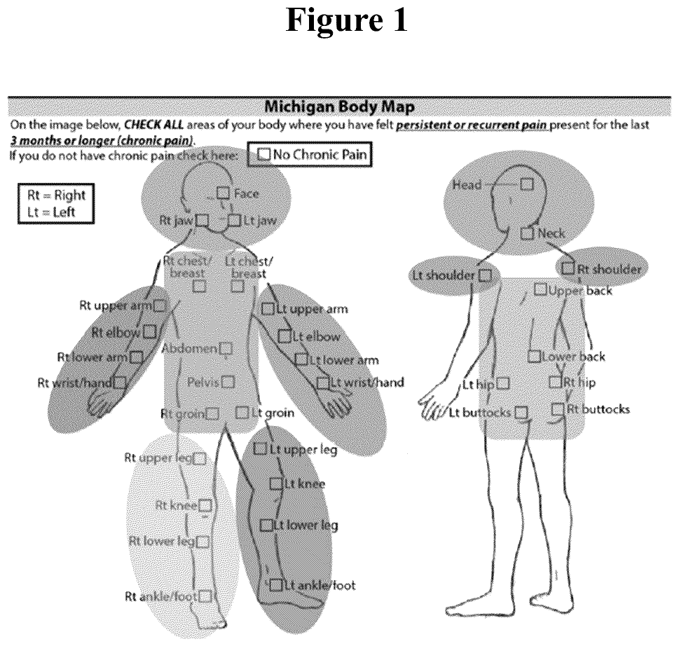

The Michigan Body Map (MBM), based on the 2011 fibromyalgia (FM) Survey Criteria, is a tool used to assess for the presence of multi-site pain in fibromyalgia. The 2011 FM Survey Criteria include the assessment of pain in 19 specific body areas using the Widespread Pain Index (WPI). The areas from the WPI are then combined with the Symptom Severity scale to assess the presence and severity of FM (Wolfe et al. Arthritis Care Res. 2010; 62(5):600-10, Wolfe et al. J Rheumatol. 2011; 38(6)1113-22). The MBM is a graphic mannequin with the 19 areas from the WPI superimposed upon it in anatomically relevant locations. The MBM also contains 16 additional areas for more general use and has been validated in patients with chronic pain (Brummett et al. Pain. 2016; 157(6):1205-12, Hassett et al. Reg Anesth Pain Med. 2019; rapm-2019-101084). A version of the MBM with the 35 areas grouped into 7 body regions is used to assess the widespreadedness of pain in subjects with PASC, with multi-site pain being defined as pain in at least 4 out of 7 regions persisting for at least 3 months (FIG. 1).

### Sheehan Disability Scale (SDS)

The Sheehan Disability Scale (SDS) is a brief self-reporting tool that rates the extent to which work/school, social life, and home life or family responsibilities are impaired by the symptoms on a 10-point visual analogue scale (Williams et al. Handbook of Psychiatric Measures. 2000). The 3 items can also be summed into a single dimensional measure of global functional impairment that ranges from 0 (unimpaired) to 30 (highly impaired).

### Daily 24-Hour Pain Recall Using 11-Point Numerical Rating Scale (NRS)

The Numerical Rating Scale (NRS) is a numeric assessment of worst pain severity, worst sleep quality, worst fatigue severity and worst memory/concentration problems within 24-hour recall using an 11-point scale ranging from 0 (no pain) to 10 (worst possible pain).

### Patient-Reported Outcome Measurement Information System (PROMIS) Scales

Patient-Reported Outcome Measurement Information System (PROMIS) is a National Institutes of Health (NIH) funded initiative to develop instruments to be used across chronic conditions (www.nihpromis.org). Three PROMIS scales include the PROMIS-Sleep disturbance scale, the PROMIS-Fatigue scale, and the PROMIS-Cognitive function scale. The scales provide questions for assessing sleep quality, severity of fatigue and cognitive function abilities, respectively, over the past 7 days using a 5-point scale ranging from 1 (not at all) to 5 (very much).

### Patient Global Impression of Change (PGI-C)

The Patient Global Impression of Change (PGI-C) is a validated instrument used to gauge the subject's assessment of change in condition (Guy. DHEW Pub No. ADM76-338 (1976), Dworkin et al. J Pain. 2008; 94:149-58). The PGI-C form provides a single question: Since the start of the study, overall my PASC is:
1=Very much improved
2=Much improved
3=Minimally improved
4=No change
5=Minimally worse
6=Much worse
7=Very much worse.

### Post-COVID-19 Functional Status (PCFS) Scale

The Post-COVID-19 Functional Status (PCFS) scale is an ordinal scale for assessment of patient-relevant functional limitations over time after COVID-19 infection (Klok et al. Eur Respir J. 2020; 56(1):2001494, Machado et al. Health Qual Life Outcomes. 2021; 19:40). The scale rates the functional status of subjects as:
0=No functional limitations
1=Negligible functional limitations
2=Slight functional limitations
3=Moderate functional limitations
4=Severe functional limitations
D=Death.

### Insomnia Severity Index (ISI)

The Insomnia Severity Index (ISI) is a 7-item self-reported questionnaire assessing the nature, severity, and impact of insomnia (Spielman et al. Sleep. 1987; 10(1):45-56, Morin et al. Sleep. 2011; 34(5):601-8). The usual recall period is the “last month” and the dimensions evaluated are severity of sleep onset, sleep maintenance and early morning awakening problems, sleep dissatisfaction, interference of sleep difficulties with daytime functioning, noticeability of sleep problems by others and distress caused by the sleep difficulties. The scores range from 0 (no problem) to 4 (very severe problem) yielding a total score ranging from 0 to 28 which is interpreted as:
0-7=Absence of insomnia
8-14=Sub-threshold insomnia
15-21=Moderate insomnia
22-28=Severe insomnia.

### Epworth Sleepiness Scale (ESS)

The Epworth Sleepiness Scale (ESS) is a self-administered questionnaire with 8 questions (Johns. Sleep. 1991; 14(6):540-5). The subject rates from 0 to 3 their usual chances of dosing off or falling asleep while engaged in 8 different activities. The total score can range from to 24, with higher rating indicating higher average sleep propensity in daily life.

In order for this application to be more fully understood, the following examples are set forth. These examples are for the purpose of illustration only and are not to be construed as limiting the scope of the application in any way. The practice of the application is illustrated by the following non-limiting examples.

### EXAMPLES

### Example 1. Study Design of Retrospective Electronic Health Record Review of Clinical Features of Subjects with PASC

### Subject Selection

A retrospective observational electronic health record (EHR) review was performed to evaluate the clinical features of complex multi-site pain, fatigue, and insomnia in subjects with Post-Acute Sequelae of Severe Acute Respiratory Syndrome (SARS)-CoV-2 infection (PASC), also referred to as long COVID or long haulers. These clinical features included (1) incidence of multi-site pain symptoms with and without associated symptoms of fatigue and insomnia, (2) association with somatic and CNS symptoms, and (3) medication use. The source of the EHRs was the TriNetX Dataworks USA Network, containing data for 75.2 million subjects. Subjects with PASC were selected based on a previously developed identification algorithm (Taquet et al. PLoS Med. 2021; 18(9): e10003773) with the following clinical features at 90 days post infection: chest/throat pain, abnormal breathing, abdominal symptoms, fatigue/malaise, anxiety/depression, pain, headache, cognitive dysfunction and myalgia and at least 1 healthcare encounter ≥180 days following the first indication of COVID-19 from the database (Table 1).

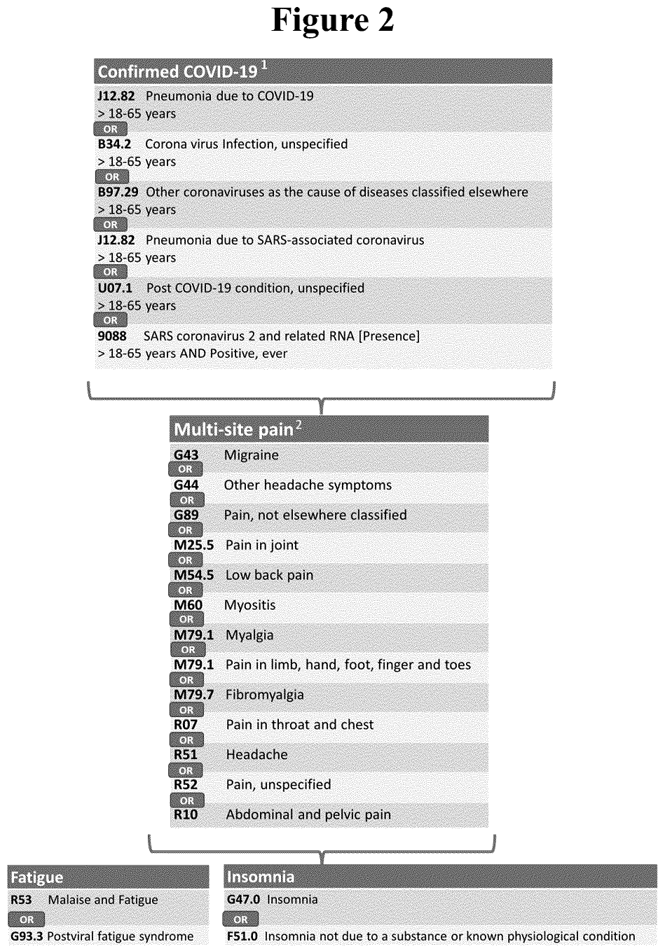

Diagnostic codes were used to capture subjects having complex pain, i.e., likely to have centrally mediated, nociplastic components. The selection of the population of interest was limited by the use of existing diagnostic codes (FIG. 2). Subjects having diagnoses associated with diffuse pain or >2 anatomically distinct sources of pain (i.e., multi-site pain) were selected by the algorithm (FIG. 2).

TABLE 1
Subject selection

| | Patients | | HCOs |
|---|---|---|---|
| NETWORK | 75,241,815 | | 48 |
| COVID-19 diagnosis or +ve PCR test; age 18-65 years | 1,004,258 | (-99%) | 47 |
| Analysis population | 1,004,258 | (0%) | 47 |
| Subjects with no other viral infections documented | 931,837 | (-7%) | 47 |
| Subjects with ≥1 HCP encounter ≥180 days post infection | 260,082 | (-72%) | 47 |
| Subjects with long-haul COVID-19 symptoms days 91-180 | 52,322 | (-80%) | 45 |

COVID = coronavirus disease;
HCO = healthcare organization;
HCP = healthcare provider;
PCR = polymerase chain reaction

### Initial Study Population

The initial study population of SARS-CoV-2-infected adults having at least 6 months of follow-up included 260,082 records, of which 52,322 met the criteria for PASC (Table 1). The data were separated into 5 subgroups: (1) subjects without multi-site pain, (2) subjects with multi-site pain but no insomnia or fatigue, (3) subjects with multi-site pain and fatigue (but no insomnia), (4) subjects with multi-site pain and insomnia (but no fatigue), and (5) subjects with multi-site pain, fatigue, and insomnia (Table 2).

TABLE 2
Subject Populations

| Subjects with PASC, aged 18-65 years, ≥180 | N = 52,322 |
|---|---|
| days follow-up | |
| Subjects with PASC without multi-site pain | 30,628 (58.5%) |
| Subjects with PASC with multi-site pain | 21,694 (41.5%) |
| Multi-site pain, no fatigue, no insomnia¹ | 17,160 (79.1%) |
| Multi-site pain + fatigue¹ | 2,782 (12.8%) |
| Multi-site pain + insomnia¹ | 1,314 (6.1%) |
| Multi-site pain + fatigue + insomnia¹ | 438 (2.0%) |

¹Cohorts are mutually exclusive;
PASC = Post-Acute Sequelae of SARS-CoV-2 infection

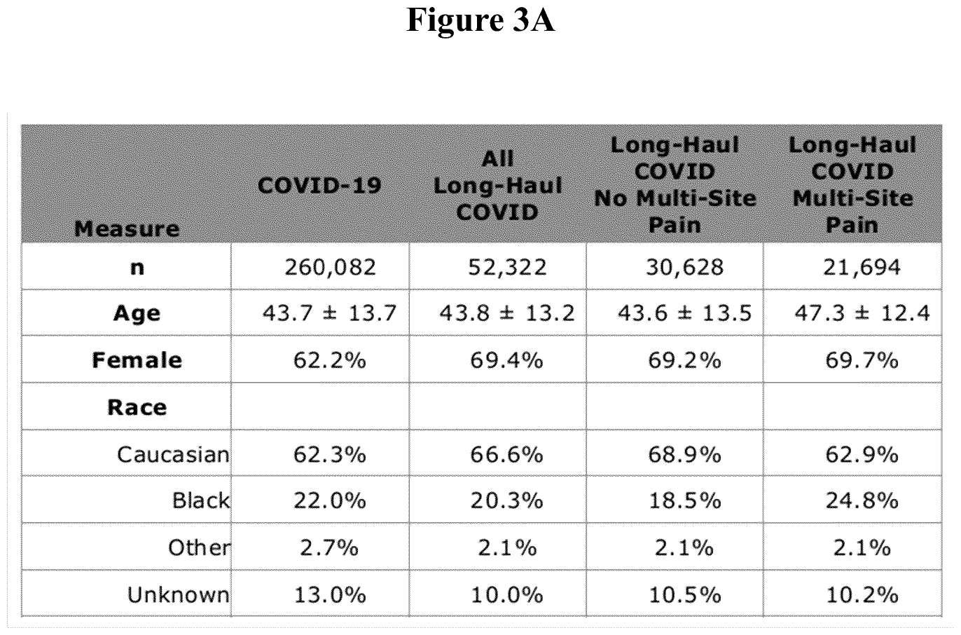

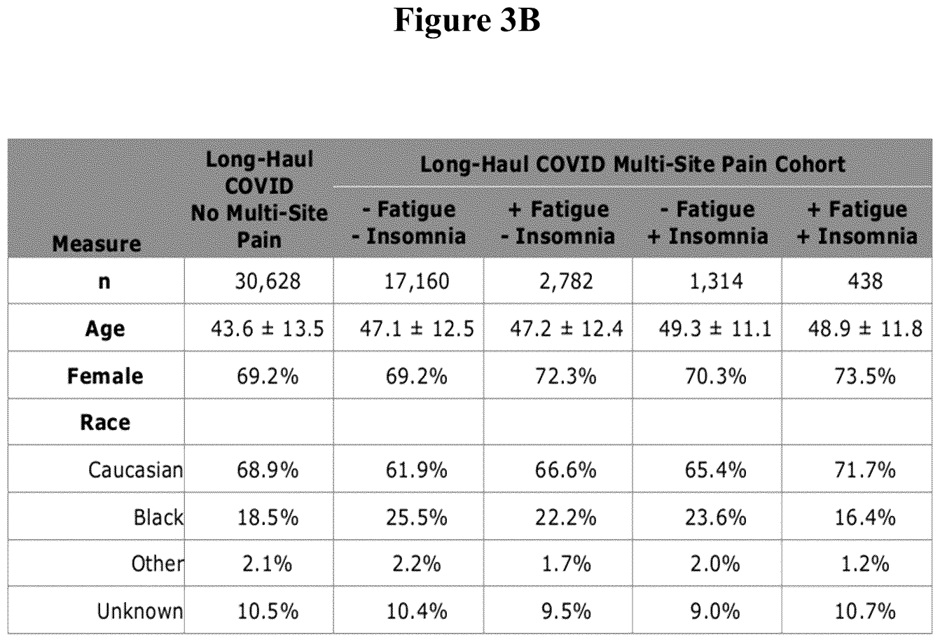

Analysis showed that PASC symptoms were present predominantly in the female population (FIG. 3A). Multi-site pain in PASC followed a similar track with a strong female preponderance (FIG. 3A). African Americans comprised 20.3% of the PASC population (FIG. 3B). The proportion of African Americans among PASC subjects was higher than in the general population, and increased in the multi-site pain groups, except in the triad of pain, fatigue and insomnia (FIG. 3B). This disparity may have public health impact, given the undertreatment and misdiagnosis of African Americans presenting with pain symptoms.

Analysis of Somatic and CNS Symptoms in Subjects with PASC

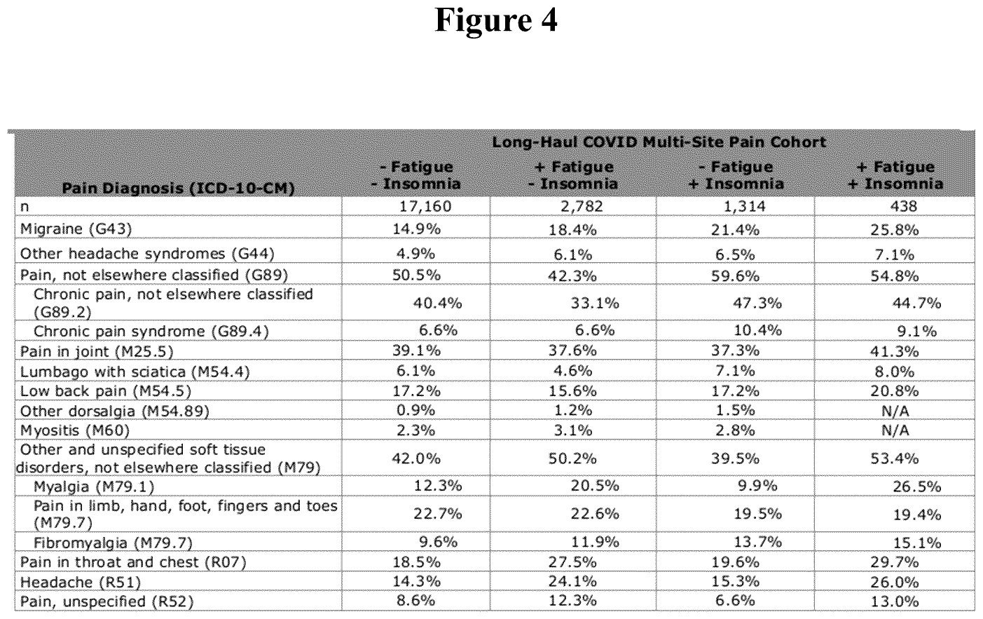

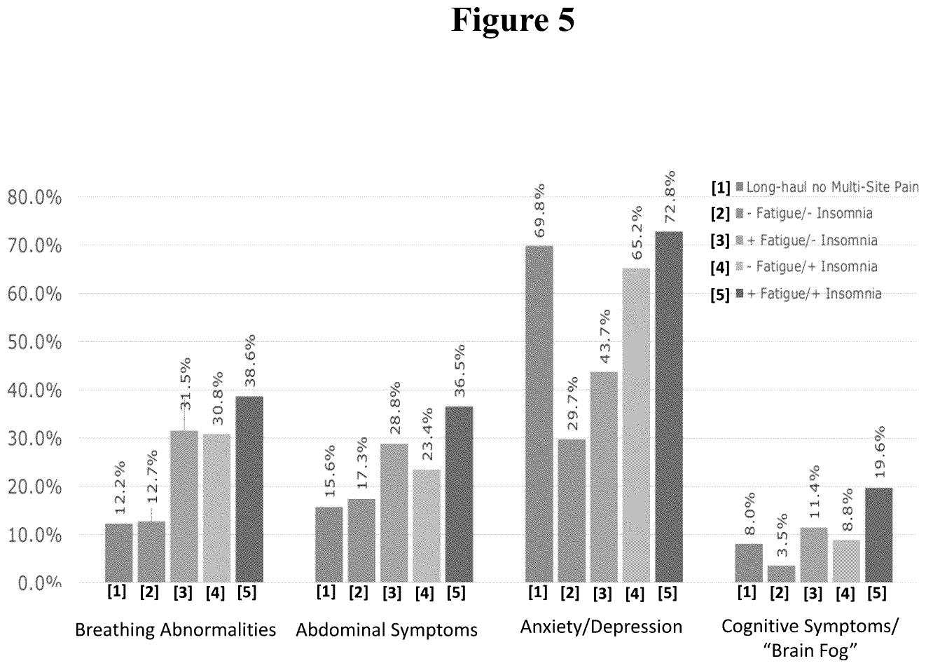

Somatic symptoms associated with PASC, such as breathing and abdominal abnormalities, occurred with approximately equal frequencies in subjects with and without multi-site pain (FIG. 5). The presence of insomnia and fatigue increased the prevalence of these symptoms and symptoms associated with a pain diagnosis (FIGS. 4 and 5).

Anxiety and depression were highly prevalent in most PASC populations, reaching almost 70% in the present study population (FIG. 5). Subjects with multi-site pain reported conspicuously lower symptoms of depression and anxiety, which may be a manifestation of somatization (nociplasticity), in which central processing of distress signals may be differentially interpreted either as depression/anxiety or as pain in different subjects (Fitzcharles et al. Lancet. 2021; 397:2098-110). Cognitive symptoms (e.g. “brain fog”) were also experienced in PASC subjects; however, they were difficult to capture within the present database due to constraints of available diagnostic codes. Cognitive symptoms exhibited a similar pattern to depression and anxiety in patients with multi-site pain (FIG. 5). As with depression and anxiety, this may reflect differential processing of central distress signals.

Inflammation of Tissue Damage Markers in Subjects with PASC

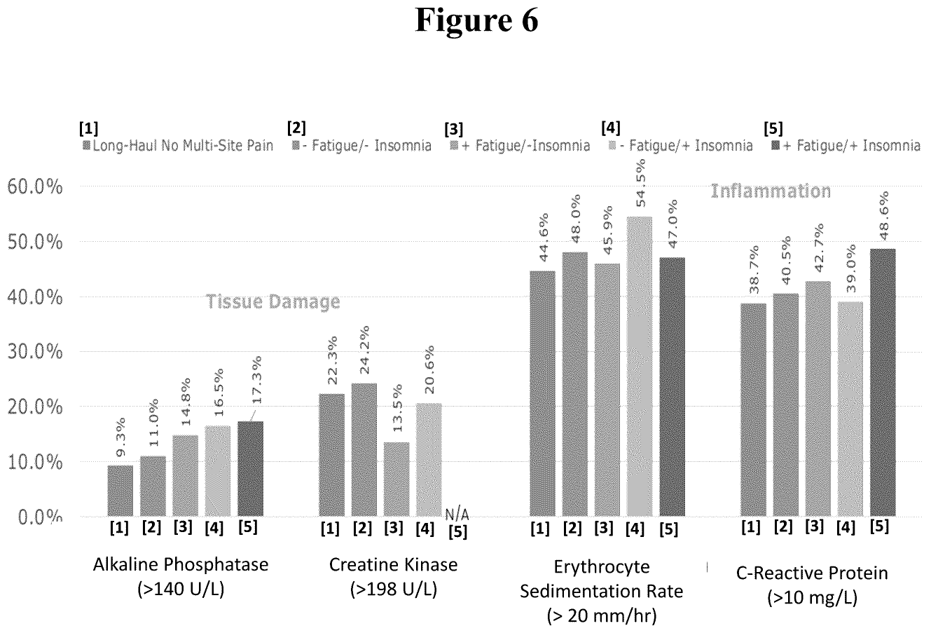

High proportions of subjects with PASC showed markers of inflammation (e.g., erythrocyte sedimentation rate and C-Reactive Protein) (FIG. 6). Overall laboratory findings did not suggest that inflammation or tissue damage are major mechanisms that contribute to multi-site pain in PASC (FIG. 6), supporting a central sensitization/nociplasticity mechanism underlying multi-site pain.

Analgesic Use in Subjects with PASC

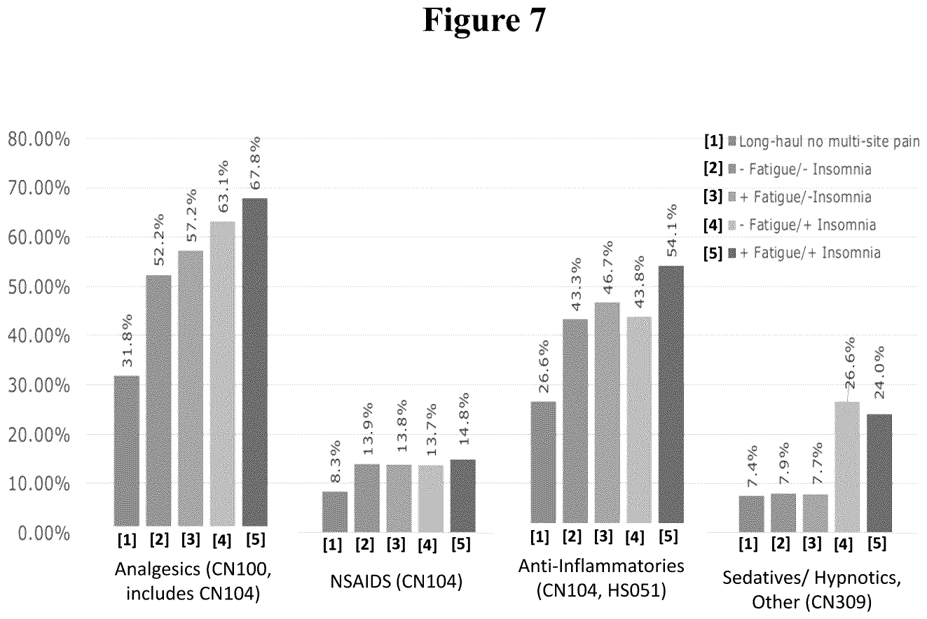

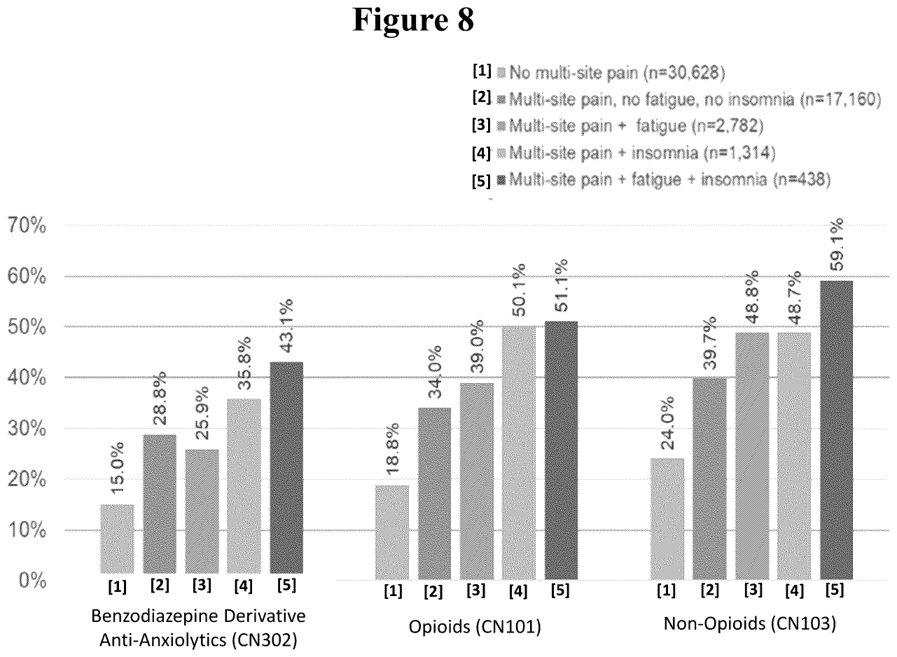

The use of analgesics, anti-inflammatory drugs and benzodiazepine anxiolytics increased with the occurrence of either fatigue or insomnia, while the use of non-steroidal anti-inflammatory drugs (NSAIDS) was consistent among all groups (FIG. 7). Opioid use in PASC patients without multi-site pain was relatively high at approximately 19% (FIG. 8). However, opioid use nearly doubled in patents with multi-site pain (FIG. 8). The occurrence of fatigue or insomnia were associated with even greater opioid use, with insomnia being an especially strong factor, resulting in >50% opioid use (FIG. 8).

### Example 2. Study Design to Evaluate TNX-102 SL Cyclobenzaprine HCl and PASC

A Phase 2, randomized, multicenter, parallel-group, double-blind, placebo-controlled, 14-week study to evaluate the efficacy and safety of TNX-102 SL 5.6 mg taken once daily (typically in two simultaneous 2.8 mg doses) at bedtime for the management or treatment of multi-site pain associated with PASC are conducted.

Approximately 470 subjects between the ages of 18-65 years are enrolled in this study. Subjects must have a positive polymerase chain reaction (PCR)-confirmed history of SARS-CoV-2 infection in the past 3 months prior to enrollment and meet the criteria for multi-site pain as defined by the Michigan Body Map closely following the SARS-CoV-2 infection, i.e., multi-site pain (defined as pain in at least 4 regions), and symptoms present at a similar level for at least 6 weeks but no longer than 12 months, with new onset or significant worsening of pain coinciding with prior COVID-19 infection. Subjects are randomized in a 1:1 ratio, i.e., 235 subjects in each of the TNX-102 SL and placebo arms.

The study consists of a Screening Visit (Visit 1), a Washout and Screening period of at least 7 days (for subjects not requiring washout) and no more than 35 days, inclusive of a 7-day baseline data collection phase immediately preceding the Baseline visit. Eligible subjects who provide written informed consent have study assessments performed at Screening and stop all excluded medications during the washout period, which must be accomplished so that the subject is medication-free for at least 14 days prior to randomization. The Screening Period is followed by a Baseline and Randomization Visit (Visit 2), and 4 treatment visits at Weeks 2, 6, 10 and 14 (Visits 3, 4 5 and 6) for efficacy and safety assessments, and assessments of study drug compliance and tolerability. There is an additional safety follow-up call at Week 16 (Visit 7). The total duration of the study for each individual is 20 weeks. The maximum treatment duration is 14 weeks.

During the Screening Visit (Visit 1), subjects are trained on the use of the diary system. Each evening, when the subject utilizes the diary, the system prompts the subject to reflect on the past 24 hours and record their worst pain severity, worst memory/concentration problems, worst fatigue, assessment of sleep quality from the previous evening, and study drug dosing the previous night (post-randomization).

Down-titration and discontinuation of excluded medications are accomplished during the Washout and Screening period immediately preceding the Baseline Visit. Following 7 days off excluded medication, subjects start the 7-day run-in Phase, during which critical baseline daily diary efficacy data are collected. Subjects are asked to record their worst daily pain severity on the 11-point (0-10) NRS scale using 24-hour recall, daily worst memory/concentration problems, daily worst fatigue, and to provide an assessment of sleep quality for the previous evening, also using an 11-point NRS scale. The average of the 7 days immediately preceding Visit 2 (Baseline/Randomization Visit; Day1) serves as the Baseline pre-treatment scores.

After completing any required washout of excluded therapies and recording Baseline Diary scores for at least 7 days, subjects return to the investigative site for baseline assessments and randomization (Day1, Visit 2), and they are randomly assigned to receive TNX-102 SL or matching placebo sublingual tablets in a 1:1 ratio.

Subjects take 1 tablet of randomly assigned study drug (TNX-102 SL 2.8 mg or placebo) sublingually once daily at bedtime for Days 1-14. Following efficacy and safety assessments, and assessment of study drug compliance at Week 2 (Visit 3), the daily dose of TNX-102 SL is increased to 5.6 mg (2×2.8 mg tablets) or 2 placebo tablets taken sublingually and simultaneously daily at bedtime. Subjects continue to record their worst daily pain, daily worst memory/concentration problems, daily worst fatigue, and to provide an assessment of sleep quality from the previous evening over the next 10 weeks.

Subjects return to the clinic at Weeks 6, 10 and 14 (Visits 4, 5 and 6, respectively) for efficacy and safety assessments and assessment of study drug compliance, and an assessment of dose tolerability at the 5.6 mg dose. In scenarios in which TNX-102 SL 5.6 mg (or 2 placebo tablets) is considered intolerable due to adverse event(s) and would otherwise lead to study discontinuation, the daily dose is lowered to 1 tablet every night (TNX-102 SL 2.8 mg or 1 placebo tablet). Re-challenge with 2 TNX-102 SL 2.8 mg (i.e., 5.6 mg dose) or placebo may be attempted at a later date if/when it is deemed clinically warranted by the Investigator, or the subject may remain on the lower dose for the remainder of the study.

The primary, secondary, and exploratory efficacy endpoints and safety endpoints are described in Table 3.

Potential genetic determinants of treatment response are examined by the assessment of genetic variants in relating to the treatment outcome. A blood sample is obtained from subjects who have signed a separate informed consent form for pharmacogenomic analyses at any visit post Screening. Exome sequencing and analysis for allelic polymorphisms related to treatment response to TNX-102 SL is performed.

TABLE 3
Criteria for Evaluation

| | |
|---|---|
| Primary Efficacy | Change from Baseline in the diary NRS weekly average of daily self- |
| Endpoints | reported worst pain severity scores at the Week 14 endpoint |
| Key Secondary | Proportion of subjects with a Patient Global Impression of Change |
| Efficacy | (PGI-C) rating of “very much improved” or “much improved” at the Week |
| Endpoints | 14 endpoint |
| | Change from Baseline in the Patient Reported Outcomes |
| | Measurement Information System (PROMIS) score for sleep disturbance at |
| | the Week 14 endpoint |
| | Change from Baseline in the PROMIS score for fatigue at the Week |
| | 14 endpoint |
| | Change from Baseline in the PROMIS score for cognitive function |
| | at the Week 14 endpoint |
| Secondary | Change from Baseline to Week 14 in the Sheehan Disability Scale |
| Efficacy | Change from Baseline in the weekly average of the daily diary y |
| Endpoints | NRS assessment of sleep quality at the Week 14 endpoint |
| | Change from Baseline in the weekly average of the daily diary NRS |
| | assessment of worst fatigue severity scores at the Week 14 endpoint |
| | Change from Baseline in the weekly average of the daily diary NRS |
| | assessment of worst memory/concentration problems severity scores at the |
| | Week 14 endpoint |
| | Change from Baseline to Week 14 in the Insomnia Severity Index |
| | Change from Baseline to Week 14 in the Epworth Sleepiness Scale |
| Exploratory | Change from Baseline in the in-clinic pain assessment - 24-hour |
| Efficacy | recall |
| Endpoints | Change from Baseline in the in-clinic pain assessment - weekly |
| | recall |
| | Proportion of subjects with ≥30% improvement worst pain severity |
| | from Baseline to Weeks 1-14 in the daily self-reported worst pain severity |
| | score |
| | Proportion of subjects with a ≥50% improvement from Baseline to |
| | Weeks 1-14 in the daily self-reported worst pain severity scores |
| | Proportion of subjects with a PGI-C rating of “very much improved” |
| | or “much improved” at each post-randomization clinic visit |
| | Change from Baseline in the PROMIS score for sleep disturbance (at |
| | each post-randomization clinic visit) |
| | Change from Baseline in the PROMIS score for fatigue (at each post- |
| | randomization clinic visit) |
| | Change from Baseline in the PROMIS score for cognitive function |
| | (at each post-randomization clinic visit) |
| | Change from Baseline in the weekly average of daily diary |
| | assessment of sleep quality at Weeks 1-14 |
| | Change from Baseline in the weekly average of daily diary |
| | assessment of worst pain severity scores at Weeks 1-14 |
| | Change from Baseline in the weekly average of daily diary |
| | assessment of worst fatigue severity scores at Weeks 1-14 |
| | Change from Baseline in the weekly average of daily diary |
| | assessment of worst memory/ concentration problems severity scores at |
| | Weeks 1-14 |
| | Change from Baseline in laboratory markers of inflammation |
| | Change from Baseline to Week 14 in the Post-COVID-19 Functional |
| | Status Scale (PCFS) |
| | Change from Baseline in the MBM pain regions (at each post- |
| | randomization clinic visit) |
| Safety Endpoints | Incidence of adverse events |
| | Changes from Baseline in clinical laboratory tests |
| | Changes from Baseline in vital signs |
| | Assessment of physical examination findings (including oral cavity) |
| | Monitoring suicidality using C-SSRS |

COVID = coronavirus disease;
C-SSRS = Columbia Suicide Severity Rating Scale;
MBM = Michigan Body Map;
NRS = numeric rating scale;
PGI-C = Patient global impression of change;
PROMIS = patient reported outcomes measurement information system

1. A method for treating Post-Acute Sequelae of Severe Acute Respiratory Syndrome (SARS)-CoV-2 infection (PASC) or one or more symptoms associated with said PASC, comprising administering to a subject in need or at risk thereof a pharmaceutical composition comprising a therapeutically effective amount of cyclobenzaprine or a pharmaceutically acceptable salt thereof and a pharmaceutically acceptable carrier.

2. The method according to claim 1, wherein the pharmaceutically acceptable salt of cyclobenzaprine in the pharmaceutical composition is cyclobenzaprine HCl.

3. (canceled)

4. The method according to claim 2, wherein the cyclobenzaprine HCl is in the form of a mannitol eutectic.

5. (canceled)

6. The method according to claim 4, wherein the mannitol eutectic is selected from the group consisting of a 75%±2% cyclobenzaprine HCl and 25%±2% β-mannitol eutectic, a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%±2% δ-mannitol eutectic and an inner layer of β-mannitol.

7. The method according to claim 4, wherein the pharmaceutical composition comprising the cyclobenzaprine HCl or the eutectic thereof further comprises a basifying agent.

8. The method according to claim 7, wherein the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.

9. (canceled)

10. The method according to claim 1, wherein the pharmaceutical composition comprises:
(i) between 0.1 mg and 30 mg of cyclobenzaprine or a pharmaceutically acceptable salt thereof;
(ii) between 1 mg and 20 mg of cyclobenzaprine or a pharmaceutically acceptable salt thereof;
(iii) less than 10 mg of cyclobenzaprine or a pharmaceutically acceptable salt thereof,
(iv) less than 5 mg of cyclobenzaprine or a pharmaceutically acceptable salt thereof,
(v) between about 2.8 mg to about 5.6 mg of cyclobenzaprine HCl;
(vi) about 5.6 mg of cyclobenzaprine HCl; or
(vii) about 2.8 mg of cyclobenzaprine HCl.

11.-16. (canceled)

17. The method according to claim 10, wherein the pharmaceutical composition is administered simultaneously or sequentially in two dosage units, and wherein:
(i) tithe combined amount of the cyclobenzaprine HCl in the two dosage units is about 5.6 mg; or
(ii) each dosage unit comprises about 2.8 mg of cyclobenzaprine HCl.

18. (canceled)

19. The method according to claim 1, wherein the pharmaceutical composition is administered daily.

20-21. (canceled)

22. The method according to claim 1, wherein the pharmaceutical composition is formulated for sublingual, buccal, intranasal, oral, intravenous, intramuscular, subcutaneous, inhalational, transdermal, rectal, vaginal, parenteral or palatal administration and wherein the pharmaceutical composition is formulated as a tablet, a thin film or a suppository.

23.-24. (canceled)

25. The method according to claim 1, wherein the pharmaceutical composition is administered for at least 14 weeks.

26. The method according to claim 1, wherein the subject has tested positive for SARS-CoV-2 infection at least three months prior to administration of the pharmaceutical composition.

27. The method according to claim 1, wherein the one or more symptoms associated with the PASC is neurologic, non-neurologic, systemic, or a combination thereof.

28. The method according to claim 1, wherein the one or more symptoms associated with the PASC is selected from the group consisting of fatigue, malaise, pain, muscle weakness, diaphoresis, chills, limb edema, dizziness, cognitive dysfunction, respiratory symptoms, cardiovascular abnormalities, alopecia, olfactory abnormalities, psychosocial symptoms, and abdominal symptoms.

29. The method according to claim 28, wherein:
(i) the respiratory symptoms are independently selected from the group consisting of polypnea, chest pain, cough, sputum, sore throat, throat pain, abnormal breathing, and shortness of breath;
(ii) the cognitive dysfunction is characterized by brain fog characterized by one or more of a memory problem, a concentration problem, a lack of mental clarity, or an inability to focus;
(iii) the psychosocial symptoms are independently selected from the group consisting of sleep disturbance, depression, anxiety, feelings of inferiority, and worse quality of life, and wherein the sleep disturbance is independently selected from the group consisting of insomnia, difficulty falling asleep, vivid or lucid dreams, and nonrestorative sleep;
(iv) the malaise is post-exertional malaise; or
(v) the pain is independently selected from the group consisting of multi-site pain, diffuse myalgia, arthralgia, musculoskeletal pain, headaches, facial pain, chest pain, abdominal pain, back pain, joint pain, body ache, lumbago with sciatica, low back pain, and pain in one or more of limb, hand, foot fingers, or toes.

30.-35. (canceled)

36. The method according to claim 29, wherein:
(i) the one or more symptoms associated with the PASC is multi-site pain;
(ii) the one or more symptoms associated with the PASC are multi-site pain and fatigue;
(iii) the one or more symptoms associated with the PASC are multi-site pain and insomnia; or
(iv) the one or more symptoms associated with the PASC are multi-site pain, fatigue, and insomnia.

37.-39. (canceled)

40. The method according to claim 36, wherein:
(i) the multi-site pain affects at least 4 regions of the body;
(ii) the multi-site pain regions are assessed using a Michigan Body Map; and
(iii) the multi-site pain region is selected from one or more of the regions of a Michigan Body Map including left arm, right arm, left leg, right leg, front of trunk, back of trunk, or head.

41.-42. (canceled)

43. The method according to claim 27, wherein the one or more symptoms associated with the PASC is new onset, follows initial recovery from an acute (SARS)-CoV-2 infection, persists post-(SARS)-CoV-2 infection, or persists post-discharge from in-patient care in a hospital, clinic or other medical facility following admission for (SARS)-CoV-2 infection.

44.-53. (canceled)

54. The method according to claim 1, wherein the one or more symptoms associated with the PASC is assessed by a Numerical Rating Scale (NRS), a Patient Global Impression of Change (PGI-C), a PROMIS scale, a Sheehan Disability Scale (SDS), a Post-COVID-19 Functional Status (PCFS) scale, an Insomnia Severity Index (ISI), an Epworth Sleepiness Scale (ESS), or a combination thereof.

55. (canceled)

56. The method according to claim 1, wherein the subject is human.
